(19) United States
(12) Patent Application Publication
(10) Pub. No.: US 2004/0166092 A1
(43) Pub. Date: Aug. 26, 2004

(54) Use of viral vectors and charged molecules for gene therapy

(76) Inventors: Francis Tufaro, Vancouver (CA); Sonia Yeung, Vancouver (CA); Brian Horsburgh, Vancouver (CA)

Correspondence Address: CLARK & ELBING LLP, 101 FEDERAL STREET, BOSTON, MA, 02110, US

(21) Appl. No.: 10/787,598

(22) Filed: Feb. 26, 2004

Related U.S. Application Data
(63) Continuation of application No. 09/592,007, filed on Jun. 12, 2000, now abandoned.
(60) Provisional application No. 60/138,875, filed on Jun. 11, 1999.

(51) Int. Cl. C12N 15/86; C12N 15/867

(52) U.S. Cl. 424/93.2; 435/456

(57) ABSTRACT

The invention provides viral vectors and charged molecules for use in gene therapy.

### CROSS-REFERENCE TO RELATED APPLICATIONS

This application is a continuation of U.S. Ser. No. 09/592,007, filed Jun. 12, 2000, which claims priority from U.S. Ser. No. 60/138,875, filed Jun. 11, 1999. Each of the prior applications are incorporated by reference herein in their entirety.

### BACKGROUND OF THE INVENTION

This invention relates to the use of viral vectors and charged molecules for gene therapy.

Skeletal muscle is an ideal seeding site for the treatment of primary myopathies or diseases requiring production of circulating proteins, because it is highly vascular and is an excellent secretory organ, with many accessible sites (Blau et al., New Eng. J. Med. 333(23):1554-1546, 1995; van Deutekom et al., Neuromuscular Disorders 8(3-4):135-148, 1998; Howell et al., Human Gene Therapy 9(5):629-934, 1998; Isaka et al., Nature Med. 2(4):418-423, 1996; Pauly et al., Gene Therapy 5(4):473-480, 1998; Bohl et al., Human Gene Therapy 8(2):195-204, 1997; Takeda, Nippon Rinsho—Japanese J. Clin. Med. 55(12):3114-3119, 1997; Tsurumi et al., Circulation 96(9 Suppl.):II-382-328, 1997). Moreover, the post-mitotic nature and longevity of muscle fibers permits stable expression of transferred genes, even if they are not integrated into chromosomal DNA (Svensson et al., Mol. Med. Today 2(4):166-172, 1996; van Deutekom et al., Mol. Med. Today 4(5):214-220, 1998). Also, high level gene expression in a relatively small number of muscle fibers may be adequate to treat inherited or acquired metabolic disorders, or to induce an immune response sufficient for vaccination (Davis et al., Human Mol. Gen. 2(11):1847-1851, 1993).

Gene transfer to skeletal muscles has been hampered in part due to the inability of current generation vectors to infect a significant number of cells (Acsadi et al., Nature 352(6338):815-818, 1991; Karpati et al., Muscle & Nerve 16(11):1141-1153; Smith et al., Nature Genetics 5(4):397-402, 1993; Acsadi et al., Human Mol. Gen. 3(4):579-584, 1994; Yang et al., Proc. Natl. Acad. Sci. U.S.A. 91(10):4407-4411, 1994; Dai et al., Proc. Natl. Acad. Sci. U.S.A. 92(5):1401-1405, 1995; Huard et al., Exp. & Mol. Path. 62(2):131-143, 1995; Mulligan, Science 260(5110):926-932, 1993). Although adeno-associated virus (AAV) efficiently infects muscle and elicits sustained gene expression, its capacity for delivering and regulating large genes is limited. As for the large DNA viruses, such as Herpes Simplex virus (HSV) and adenovirus, muscle fibers exhibit a maturation-dependent loss of susceptibility to infection (Acsadi et al., Human Mol. Gen. 3(4):579-584, 1994; Huard et al., Human Gene Therapy 8(4):439-452, 1997; Feero et al., Human Gene Therapy 8(4):371-380, 1997; Huard et al., Neuromuscular Disorders 7(5):299-313, 1997; Huard et al., J. Virol. 70(11):8117-8123, 1996; Huard et al., Gene Therapy 2(6):385-392, 1995; Inui et al., Brain & Dev. 18(5):357-361, 1996; Ragot et al., Nature 361(6413):647-650, 1993; Quantin et al., Proc. Natl. Acad. Sci. U.S.A. 89(7):2581-2584, 1992; Vincent et al., Nature Genetics 5(2):130-134, 1993). Previous studies of HSV infection in rodents show that the loss of infectivity may be due, at least in part, to the development of the basal lamina throughout the course of maturation, which may block the initial events in HSV infection (Huard et al., J. Virol. 70(11):8117-8123, 1996).

Cancer is another disease for which many therapies are being tested. One area of intense research in the cancer field is gene therapy. Cancer gene therapy suffers from many of the same problems as the muscle gene therapies described above. For example, the current vectors cannot be delivered accurately to a sufficient number of cells to reduce tumor growth and increase patient survival.

To initiate infection, HSV attaches to cell surface glycosaminoglycans, such as heparan sulfate and dermatan sulfate (Spear et al., Adv. Exp. Med. & Biol. 313:341-353, 1992; Shieh et al., 116(5):1273-1281, 1992; Fuller et al., J. Virol. 66(8):5002-5012, 1992; Gruenheid et al., J. Virol. 67(1):93-100, 1993; Herold et al., J. Gen. Virol. 75(6):1211-1222, 1994; Banfield et al., Virology 208(2):531-539, 1995; Williams et al., J. Virol. 71(2):1375-1380, 1997), which stabilize the virus such that it can interact with secondary protein receptors required for entry into host cells (Terry-Allison et al., J. Virol. 72(7):5802-5810, 1998; Geraghty et al., Science 280(5369):1618-1620, 1998; Montgomery et al., Cell 87(3):427-436, 1996).

### SUMMARY OF THE INVENTION

The invention provides methods for introducing vectors (e.g., viral vectors, such as HSV vectors) into cells by co-administration of the vectors with a charged molecule (e.g., a charged polysaccharide, such as a glycosaminoglycan or analog thereof). The methods of the invention can be used to introduce genes into cells for use in gene therapy or vaccination.

Accordingly, the invention features methods for introducing a nucleic acid vector into a living cell, by contacting the cell with the vector and, either before, during, or after this contacting, contacting the cell with a liquid medium comprising a compound that, in the medium, is charged, non-cytotoxic, and capable of facilitating the uptake of the vector by the cell. Preferably this method is carried out in a mammal, for example, a human patient. Vectors that can be used in the methods of the invention use glycosaminoglycans as receptors or co-receptors for entry into cells. For example, viral vectors of the family Herpesviridae (e.g., HSV-1, HSV-2, VZV, CMV, EBV, HHV6, and HHV7), as well as Dengue virus, Adeno-associated virus (AAV), Adenovirus, papillomavirus, and retrovirus (e.g., lentivirus, such as HIV)-based vectors can be used. Also, bacterial vectors, such as Listeria monocytogenes-based vectors can be used. Preferably, the vectors are attenuated, and examples of attenuated viral vectors that can be used in the invention are provided below.

Molecules carrying either a negative or positive charge that can be used in the invention include charged polysaccharides, such as glycosaminoglycans and analogs thereof, polylysine, acyclodextrin, and diethylaminoethane (DEAE). Examples of glycosaminoglycans and glycosaminoglycan analogs that can be used in the invention include, for example, dextran sulfate, dermatan sulfate, heparan sulfate, chondroitin sulfate, and keratin sulfate. An additional charged molecule that can be used in the invention is polyethylene glycol. As is discussed further below, the charged molecules can be administered prior to, or concurrent with, the vectors in the methods of the invention.

Cells that may be used in the invention include mature muscle cells, retinal cells, and cancer cells.

Conditions and diseases for which the method can be used include cancer, primary myopathies, and conditions and diseases that can be treated by production of a therapeutic product into circulation. Specific examples of these conditions and diseases, as well as genes that can be included in vectors to effect their treatment, such as genes encoding polypeptides (for example, growth factors, enzymes, anti-angiogenic polypeptides, and polypeptides that promote cell death), hormones, vaccine antigens, antisense molecules, and ribozymes, are described further below. In addition, the vector and charged molecule may be delivered to the subject locally or systemically.

The invention provides many advantages. For example, glycosaminoglycans and glycosaminoglycan analogs, such as dextran sulfate, are non-destructive, non-toxic, and limit the spread of viral vectors to other sites in the tissue. The methods of the invention, thus, represent an approach for targeted expression of genes in HSV vectors in desired cells or tissues by direct injection. Also, HSV is attractive as a gene delivery vector, because its large size allows for the delivery of several large genes at once, and it can be made relatively non-toxic (Huard et al., Neuromuscular Disorders 7(5):299-313, 1997; Glorioso et al., Annual Rev. Microbiol. 49:675-710, 1995). Moreover, HSV can be grown to high titers, can infect non-dividing cells efficiently (Lim et al., Biotechniques 20(3):460-469, 1996), and can be controlled through the action of antiviral drugs, such as acyclovir, that inactivate virus replication (Evrard et al., Cell Biol. & Toxic. 12(4-6):345-350, 1996; Black et al., Proc. Natl. Acad. Sci. U.S.A. 93(8):3525-3529, 1996; Hasegawa et al., Am. J. Resp. Cell & Mol. Biol. 8(6):655-661, 1993). Non-replicating HSV vectors also are relatively non-toxic, and thus can contribute to alleviation of the immunogenicity of foreign protein expression in tissues. Finally, as is noted above, skeletal muscle is an ideal site for the treatment of myopathies and other disorders, as it is highly vascular and is an excellent secretory organ, with many accessible sites.

Other features and advantages of the invention will be apparent from the following detailed description and the drawings.

### BRIEF DESCRIPTION OF THE DRAWINGS

FIG. 1 is a photograph of HSV ICP4 Immunofluorescence showing infected nuclei of mature myofibers. Isolated myofibers infected with G207 were processed for indirect immunofluorescence using a mouse anti-ICP4 antibody. (a) G207 only, mature myofiber; (b) G207 only, immature myofiber; (c) G207 only; (d) G207+0.33 mg/ml collagenase type IV; (e) G207+3 μg/ml dextran sulfate; (f) G207+10 μg/ml dextran sulfate; (g) G207+2 U/ml chondroitin ABC lyase; (h) G207+4 U/ml chondroitin ABC lyase. Magnification: a, b, c, e-g×20; h×40; d×60. Images were captured by confocal microscopy: c-g.

FIG. 2 is a graph showing anion-exchange HPLC of cell-associated glycosaminoglycans derived from myofibers belonging to different age-groups. Myofibers were labeled with [³⁵S] sulfate for 24 hours. The medium was removed, and the monolayers were washed extensively to remove any traces of medium from the cells. Glycosaminoglycans were isolated and fractionated by HPLC. HS, elution position of heparan sulfate; CS, elution position of chondroitin sulfate. Open diamonds, myofibers isolated from 8-day old mice; closed diamonds, myofibers isolated from 2-month old mice. The dotted line represents the salt gradient used for elution.

FIG. 3 is a photograph showing the results of in vivo injection of immature skeletal muscle with G207. Cryostat sections of immature mouse TA muscle taken 3 days post-injection of HSV and stained histochemically for β-galactosidase. Gene transfer was carried out by intramuscular injection of G207 (1×10⁶ plaque forming units (pfu)) in a volume of 50 μl. (a) & (b) G207 only. Magnification: a×10, b×40.

FIG. 4 is a photograph showing infection of mature skeletal muscle with G207. β-galactosidase gene transfer to skeletal muscle of adult mice. G207 (1×10⁶ pfu) and the proposed treatments were co-injected into the tibialis anterior of 2-month old balb/c mice in a total injection volume of 50 μl. Frozen sections were cut and stained for β-galactosidase activity. (a) & (b) G207 only; (c) & (d) G207+0.33 mg/ml collagenase type IV; (e) & (f) G207+10 μg/ml dextran sulfate; (g) & (h) G207+2 U/ml chondroitin ABC lyase. Magnification: a, c, e, g×20; b, d, f, h×40.

FIG. 5 is a set of photographs showing that HSV reaches tumor tissue after systemic delivery. HSV was administered to mice by tail vein, reaching a distant flank tumor (left panel), or locoregionally by portal vein, reaching a liver tumor (right panel). The tumor tissues were stained for β-galactosidase to detect cells infected with HSV.

FIG. 6 is a schematic showing injection of labeled HSV in a mouse (right panel) and a graph of viral particle delivery to various tissues in mice with flank tumors. Mice were injected with 1×10⁷ pfu of ³⁵S methionine-labeled NV1020. Two hours later, the animals were sacrificed and their tissues were measured for the presence of viral particles.

FIG. 7 is a graph showing the effect of systemic delivery of NV1020 (HSV) on mouse survival in a CT-26 liver metastatic cancer model. Mice with liver tumor nodules were systemically delivered 1×10⁷ pfu of NV1020 or PBS (control). The percent of surviving mice was measured over time.

FIG. 8 is a graph showing the effect of intratumoral or systemic delivery of NV1020 or NV1020 plus F1 (dextran sulfate) on anti-tumor efficacy in a CT-26 flank tumor model. Mice with flank tumors were administered NV1020 either intratumorally (IT) or systemically (IV; with or without F1 (dextran sulfate)), by tail vein. Tumor growth rates were then assessed.

FIG. 9 is a graph showing the effect of dextran sulfate (DexS) dosage on anti-tumor efficacy. Mice with flank tumors were administered a dose of 1×10⁷ pfu of NV1020, 1×10⁷ pfu of NV1020 plus 10 μg/ml of dextran sulfate, 1×10⁷ pfu of NV1020 plus 100 μg/ml of dextran sulfate, 1×10⁷ pfu of NV1020 plus 500 μg/ml of dextran sulfate, or PBS plus 100 μg/ml of dextran sulfate (control), at day 0, day 2, and day 4. Tumor growth rates were then assessed.

FIG. 10 is a graph illustrating the effects of multiple HSV plus dextran sulfate (DexS) dosing on anti-tumor efficacy in a CT-26 flank tumor model. Mice with flank tumors were administered one dose of 3×10⁷ pfu of NV1020, one dose of 3×10⁷ pfu of NV1020 plus 100 μg/ml of dextran sulfate, three doses of 1×10⁷ pfu of NV1020 plus 100 μg/ml of dextran sulfate, or PBS (control). Tumor growth rates were then assessed.

FIG. 11 is a graph showing the effect of multiple dosing of NV1020 plus dextran sulfate (DexS) on mouse survival in a CT-26 flank tumor model. Mice with flank tumors were administered one dose of 3×10⁷ pfu of NV1020, one dose of 3×10⁷ pfu of NV1020 plus 100 μg/ml of dextran sulfate, three doses of 1×10⁷ pfu of NV1020 plus 100 μg/ml of dextran sulfate, or PBS (control). Mouse survival was then measured over time.

FIG. 12 is a graph showing the effect of dextran sulfate (DexS) on anti-tumor efficacy in a CT-26 liver metastatic model. Mice with metastasized tumors were administered 1×10⁷ pfu of NV1020, 1×10⁷ pfu of NV1020 plus dextran sulfate (100 μg/ml), PBS only (control), or PBS plus dextran sulfate (100 μg/ml) (control). The number of tumor nodules was assessed 13 days later.

FIG. 13 is a graph illustrating the effects of dextran sulfate and acyclovir on anti-tumor efficacy in a CT-26 flank tumor model. Mice with flank tumors were administered a dose of 1×10⁷ pfu of NV1020, 1×10⁷ pfu of NV1020 plus dextran sulfate (F1; 100 μg/ml), NV1020 plus dextran only (F2), 1×10⁷ pfu of NV1020 plus dextran sulfate and acyclovir (F3; 2 mg/ml), or PBS plus dextran sulfate (100 μg/ml) (control) at day 0, day 2, and day 4. Tumor growth rates were then assessed.

FIG. 14 is a graph showing the effect of G207 plus dextran sulfate (DexS) on anti-tumor efficacy. Mice with flank tumors were administered a dose of 1×10⁷ pfu of NV1020, 1×10⁷ pfu of G207, 1×10⁷ pfu of G207 plus dextran sulfate (100 μg/ml), or PBS (control) at day 0, day 2, and day 4. Tumor growth rates were then measured.

FIG. 15 is a set of photographs showing the effect of dextran sulfate (DexS) on CT-26 morphology and cell growth in vitro. No compound (panel A), or dextran sulfate (100 μg/ml; panel B) was added to CT-26 cells in culture. After 48 hours, cell numbers and morphology were examined.

FIG. 16 is a graph showing the effects of dextran sulfate (DexS) and acyclovir (ACV) on CT-26 growth in culture. In vitro cultured CT-26 cells were administered dextran sulfate (100 μg/ml), acyclovir, (2 mg/ml) both dextran sulfate and acyclovir, or were left untreated. Cell proliferation was measured over time.

FIG. 17 is a set of photographs showing the effect of dextran sulfate on peripheral degeneration of flank tumors. Mice were administered 1×10⁷ pfu of NV1020 (panel A) or 1×10⁷ pfu of NV1020 plus dextran sulfate (F1) (panel B). The tumor was then removed and prepared for histochemical analysis to evaluate tumor necrosis.

FIG. 18 is a graph showing the effect of dextran sulfate (DexS) on the bioavailability of virus in vivo. One×10⁷ pfu of NV1020 or 1×10⁷ pfu of NV1020 plus dextran sulfate was administered by tail vein to mice. Groups of mice were then sacrificed at varying time points. Blood samples were removed and the serum was analyzed for infectious virus.

FIG. 19 is a graph demonstrating the effect of dextran sulfate (DexS) on the in vivo distribution of NV1020 in various tissues. One×10⁷ pfu of radioactive-labeled (³⁵S) NV1020 or 1×10⁷ pfu of radioactive-labeled (³⁵S) NV1020 plus dextran sulfate (100 μg/ml) was administered to mice containing flank tumors by tail vein. After 2 hours or 12 hours, the animals were sacrificed, various organs were harvested, homogenized, and assessed for distribution of (³⁵S) NV1020.

### DETAILED DESCRIPTION

The invention provides methods for introducing vectors (e.g., viral vectors, such as HSV vectors) into cells, for example, mature skeletal muscle cells or tumor cells, by co-administering the viral vectors with a charged molecule, such as a charged polysaccharide, e.g., a glycosaminoglycan or analog thereof. The methods of the invention can be used to introduce genes into cells in vivo for the purpose of, for example, gene therapy or vaccination.

Vectors that can be used in the invention use glycosaminoglycans as receptors or co-receptors for entry into the cells. For example, viral vectors of the family Herpesviridae (e.g., HSV-1, HSV-2, VZV, CMV, EBV, HHV-6, HHV-7, and HHV-8), as well as Dengue virus, Adeno-associated virus (AAV), Adenovirus, papillomavirus, and lentivirus (e.g., HIV)-based vectors can be used. Bacterial vectors, such as Listeria monocytogenes-based vectors can also be used in the methods of the invention.

In some cases, it is desirable that viral vectors used in the invention are attenuated or mutated, so that they do not replicate in or kill the cells into which they are introduced by, for example, inducing lysis or apoptosis of the cells. In other cases, for example, in tumor cell gene therapy, it is beneficial that the vectors can replicate in a cell and kill it. Numerous appropriate mutant viruses having these characteristics are known and can readily be adapted for use in the invention by those of ordinary skill in this art. For example, in the case of HSV, the vectors of Geller (U.S. Pat. No. 5,501,979; WO 90/09441; American Type Culture Collection (ATCC), Rockville, Md., ATCC Accession Number 40544), Breakfield (EP 453,242-A1), Speck (WO 96/04395), Preston et al. (WO 96/04394), DeLuca (U.S. Pat. No. 5,658,724), and Martuza (U.S. Pat. No. 5,585,096) can be adapted for use in the methods of the invention. Specific examples of attenuated HSV mutants that can be used in the invention include NV1020 (described below), G207 (Yazaki et al., Cancer Res. 55(21):4752-4756, 1995), HF (ATCC VR-260), MacIntyre (ATCC VR-539), MP (ATCC VR-735); HSV-2 strains G (ATCC VR-724) and MS (ATCC VR-540); as well as mutants having mutations in one or more of the following genes: the immediate early genes ICP0, ICP4, ICP22, ICP27, and ICP47 (U.S. Pat. No. 5,658,724); genes necessary for viral replication, UL9, UL5, UL42, DNA pol, and ICP8; the γ34.5 gene; the ribonucleotide reductase gene; the VP16 gene (i.e., Vmw65, WO 91/02788, WO 96/04395, WO 96/04394); and the gH, gL, gD or gB genes (WO 92/05263, WO 94/21807, WO 94/03207).

Charged molecules that can be used in the invention include charged polysaccharides, such as glycosaminoglycans and analogs thereof, polylysine, acyclodextrin, and diethylaminoethane (DEAE). Examples of glycosaminoglycans and glycosaminoglycan analogs that can be used in the invention include, for example, dextran sulfate, dermatan sulfate, heparan sulfate, chondroitin sulfate, and keratin sulfate. An additional charged molecule that can be used in the invention is polyethylene glycol. As is discussed further below, the charged molecules can be administered prior to, or concurrent with, the vectors in the methods of the invention.

Conditions that can be treated using the methods of the invention include cancer, primary myopathies, as well as conditions that can be treated by the production of circulating proteins. Thus, vectors used in the methods of the invention can include one or more genes encoding one or more therapeutic gene products, such as a polypeptide, for example, a growth factor, an enzyme, a polypeptide that promotes cell death, an anti-angiogenic polypeptide, or an immunomodulatory protein, a hormone, an antisense RNA molecule, or a ribozyme (see below), expression of which will alleviate or prevent a symptom of a condition or disease. Alternatively, the gene can encode a vaccine antigen, and the method of the invention, thus, can be used to induce a prophylactic or therapeutic immune response, for example, to an undesired pathogen or cell type, such as a cancer cell.

Specific examples of conditions that can be treated using the methods of the invention (as well as corresponding genes to be included in vectors for treating the conditions) are as follows: restenosis (β-ARKct (laccarino et al., Proc. Natl. Acad. Sci. U.S.A. 96(7):3945-3950, 1999); fibroblast growth factor receptor (Yukawa et al., Atherosclerosis 141(1):125-132, 1998)); laryngeal paralysis and muscle atrophy, by enhancement of nerve sprouting and muscle re-innervation (insulin-like growth factor-1 (IGF-1) (Shiotani et al., Archives Otolaryngology 125(5):555-560, 1999)); mucopolysaccharidosis type VII (β-glucouronidase (Daly et al., Human Gene Therapy 10(1):85-94, 1999)); limb-girdle muscular dystrophies 2C-F (δ-sarcoglycan (Greelish et al., Nature Medicine 5(4):439-443, 1999)); fibrotic diseases, such as glomerulonephritis and glomerulosclerosis (transforming growth factor-β type II receptor-IgG Fc chimera (Isaka et al., Kidney Intl. 55(2):740-741, 1999)); mucopolysaccharidosis type VI (N-acetylgalactosamine 4-sulfatase (Yogalingam et al., DNA & Cell Biol. 18(3):187-195, 1999)); motor neuron diseases (neurotrophin-3 and other neurotrophic factors, such as CNTF, BDNF, and IGF-1 (Haase et al., J. Neuro. Sci. 160(Suppl. 1):S97-105, 1998)); hypertension (angiotensin II type 1 receptor antisense (Gelband et al., Hypertension 33(1):360-365, 1999)); atherosclerosis and hypercholesterolemia (apoliproptein AI and lecithin-cholesterol acyltransferase (Fan et al., Gene Therapy 5(10):1434-1440, 1998)); induction of an alloimmune response (donor MHC class I (Zhai et al., Transplant Immunology 6(3):169-175, 1998)); hemophilia (Factor VIII, Factor IX (Herzog et al., Nature Medicine 5(1):56-63, 1999; Herzog et al., Cur. Opin. Hemat. 5(5):321-326, 1998)); loss of skeletal muscle function in aging (IGF-1 (Barton-Davis et al., Proc. Natl. Acad. Sci. U.S.A. 95(26):15603-15607, 1998)); liver enzyme deficiencies (phenylalanine hydroxylase (Harding et al., Gene Therapy 5(5):677-683, 1998)); non-insulin dependent diabetes mellitus (GLUT4 (Galuska et al., Adv. Exp. Med. & Biol. 441:73-85, 1998)); glycogen storage disease (acid alpha-glucosidase (Nicolino et al., Human Mol. Gen. 7(11):1695-1702, 1998)); muscular dystrophy (dystrophin (Baranov et al., Genetika 34(7):876-882, 1998); utrophin (Rafael et al., Nature Genetics 19(1):79-82, 1998)); tumor and metastasis suppression, vaccine adjuvant, and pathogen defense (interleukin-12 (Lee et al., Human Gene Therapy 9(4):457-465, 1998)); and acute limb ischemia (vascular endothelial growth factor (Tsurumi et al., Circulation 96(Suppl. 9):II-382-8, 1997)). Additional therapeutic products that can be produced using the methods of the invention include, for example, growth hormone, erythropoietin, and insulin, immunomodulatory proteins, antiangiogenic proteins, cytokines, and polypeptides involved in cell death.

As is noted above, the therapeutic product encoded by a gene in a vector used in the methods of the invention can also be an RNA molecule, such as an antisense RNA molecule that, by hybridization interactions, can be used to block expression of a cellular or pathogen mRNA. Alternatively, the RNA molecule can be a ribozyme (e.g., a hammerhead or a hairpin-based ribozyme) designed either to repair a defective cellular RNA or to destroy an undesired cellular or pathogen-encoded RNA (see, e.g., Sullenger, Chem. Biol. 2(5):249-253, 1995; Czubayko et al., Gene Ther. 4(9):943-949, 1997; Rossi, Ciba Found. Symp. 209:195-204, 1997; James et al., Blood 91(2):371-382, 1998; Sullenger, Cytokines Mol. Ther. 2(3):201-205, 1996; Hampel, Prog. Nucleic Acid Res. Mol. Bio. 58:1-39, 1998; Curcio et al., Pharmacol. Ther. 74(3):317-332, 1997).

Genes can be inserted into vectors used in the methods of the invention using standard methods (see, e.g., Ausubel et al., Current Protocols in Molecular Biology, John Wiley & Sons, New York, N.Y., 1998). The genes can be inserted so that they are under the control of vector regulatory sequences. Alternatively, the genes can be inserted as part of an expression cassette that includes regulatory elements, such as promoters or enhancers. Appropriate regulatory elements can be selected by one of ordinary skill in this art based on, for example, the desired tissue-specificity and level of expression. For example, a tissue- or cell type-specific (e.g., muscle-specific or a tissue in which a tumor occurs) promoter can be used to limit expression of a gene product to a specific tissue or cell type. In addition to using tissue-specific promoters, local administration of the vector and/or charged molecule can be used to achieve localized expression.

Examples of non-tissue-specific promoters that can be used in the invention include the early Cytomegalovirus (CMV) promoter (U.S. Pat. No. 4,168,062) and the Rous Sarcoma Virus promoter (Norton et al., Molec. Cell Biol. 5:281, 1985). Also, HSV promoters, such as HSV-1 IE and IE 4/5 promoters, can be used. An example of a tissue-specific promoter that can be used in the invention is the desmin promoter, which is specific for muscle cells (Li et al., Gene 78:243, 1989; Li et al., J. Biol. Chem. 266:6562, 1991). Other muscle-specific promoters are known in the art, and can readily be adapted for use in the invention.

The vectors and charged molecules can be administered to a patient (e.g., a human patient) according to the methods of the invention by, for example, direct injection into a tissue, for example, a muscle or a tissue in which a tumor is present, or by surgical methods. Alternatively, administration of one or both of these agents can be parenteral, intravenous, subcutaneous, intraperitoneal, intradermal, or intraepidermal route, or via a mucosal surface, e.g., an ocular, intranasal, pulmonary, oral, intestinal, rectal, vaginal, or urinary tract surface.

Any of a number of well known formulations for introducing vectors into cells in mammals can be used in the invention (see, e.g., Remington's Pharmaceutical Sciences (18^{th} edition), ed., A. Gennaro, 1990, Mack Publishing Co., Easton, Pa.). For example, the vectors can be used in a naked form, free of any packaging or delivery vehicle. The vectors (as well as the charged molecules) can be simply diluted in a physiologically acceptable solution, such as sterile saline or sterile buffered saline, with or without a carrier.

The amount of vector to be administered depends, e.g., on the specific goal to be achieved, the strength of any promoter used in the vector, the condition of the mammal intended for administration (e.g., the weight, age, and general health of the mammal), the mode of administration, and the type of formulation. In general, a therapeutically or prophylactically effective dose of, e.g., from about 1 ng to about 1 mg, preferably, from about 10 μg to about 800 μg, is administered to human adults.

The amount of charged molecule to be administered can be determined by one of skill in this art, and can be, for example, from about 1 ng/ml to about 100 μg/ml, but, preferably, is less than 10 μg/ml. Administration of both the vector and the charged molecule can be achieved in a single dose or repeated at intervals. Also, the charged molecule can be administered concurrently with or prior to (e.g., up to five hours, such as three hours) the vector.

The methods of the invention are based on our discovery, which is described further below, that glycosaminoglycan synthesis is down-regulated during murine skeletal muscle maturation. This could account for the loss of HSV infectivity in maturing murine skeletal muscle, because heparan sulfate acts as a co-receptor for attachment of HSV to cells. (Montgomery et al., Cell 87(3):427-436, 1996; Geraghty et al., Science 280(5369):1618-1620, 1998; Whitbeck et al., J. Virol. 71(8):6083-6093, 1997). To test whether secondary HSV receptors were present, myofibers were treated with a variety of enzymes, including collagenase type IV and chondroitin ABC lyase. Both of these treatments enhanced HSV infection, which suggests that virus receptors were present, but not readily accessible to the virus in the intact myofiber. Surprisingly, we also found that infectivity of HSV-1, but not HSV type 2 (HSV-2), could be restored by exposing myofibers to low concentrations of the glycosaminoglycan analog dextran sulfate. Dextran sulfate has been shown previously to promote HSV-1, but not HSV-2, infection in the absence of heparan sulfate. This supports the hypothesis that a lack of accessible heparan sulfate is responsible for the resistance of mature myofibers to HSV-1 infection. Taken together, these results show that the basal lamina is not an absolute block to infection, and that dextran sulfate can be used as a surrogate co-receptor for the nondestructive targeting of HSV-1 to mature skeletal muscle. These findings, which are described in detail below, greatly expand the usefulness of HSV as a gene therapy vector for the treatment of inherited and acquired diseases.

We have also discovered that the infection of tumor cells by HSV is increased through co-administration of a charged molecule, for example, a glycosaminoglycan or glycosaminoglycan analog. These results, further described below, again bolster the use of HSV as a gene therapy vector in treating cancer or other cell proliferation diseases or conditions.

Results

Mature Myofibers are Refractory to Infection

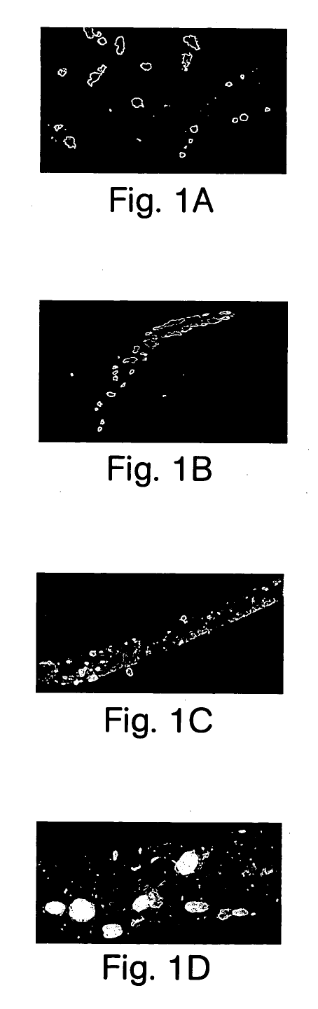

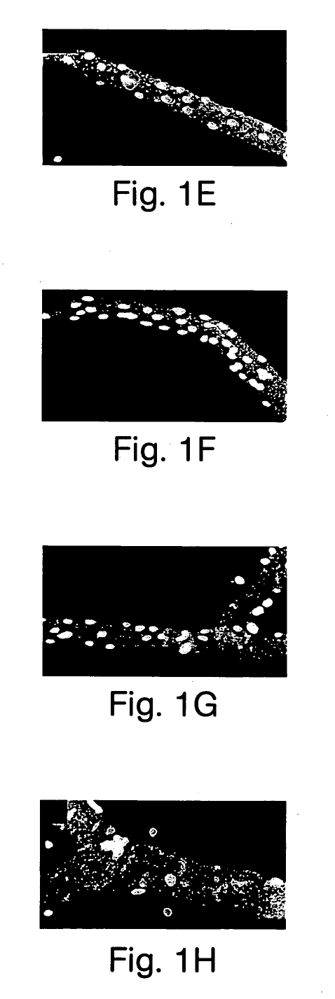

It has been shown previously that HSV vectors infect newborn muscle fibers in vitro, but not those isolated from older animals (Huard et al., Human Gene Therapy 8(4):439-452, 1997; Feero et al., Human Gene Therapy 8(4):371-380, 1997; Huard et al., Neuromuscular Disorders 7(5):299-313, 1997; Huard et al., J. Virol. 70(11):8117-8123, 1996). To investigate the underlying basis for the maturation-dependent loss of infection, single muscle fibers were established in culture, and exposed to G207, which is an attenuated replication-defective HSV-1 vector that expresses β-galactosidase following infection (Yazaki et al., Cancer Res. 55(21):4752-4756, 1995; Mineta et al., Nature Med. 1(9):938-943, 1995). In these assays, newborn myofibers were completely susceptible to infection, whereas only 6% of mature myofibers were infected at this concentration of virus (Table 1 and FIG. 1, a and b). Thus, these results were consistent with previous studies showing a maturation-dependent loss of susceptibility to HSV infection (Feero et al., Human Gene Therapy 8(4):371-380, 1997; Huard et al., J. Virol. 70(11):8117-8123, 1996).

TABLE 1
Number of myofibers isolated from mature mouse EDL
muscle that express lac Z following inoculation with G207
and proposed treatments

| | Total # | |
|---|---|---|
| Treatment | of fibers | % positive |
| G207 only | 88 | 6 |
| 0.02 mg/ml collagenase type IV | 68 | 7 |
| 0.20 mg/ml collagenase type IV | 81 | 31 |
| 0.33 mg/ml collagenase type IV | 77 | 97 |
| 0.66 mg/ml collagenase type IV | 67 | 0* |
| 0.3 μg/ml dextran sulfate | 78 | 6 |
| 3.0 μg/ml dextran sulfate | 75 | 7 |
| 10 μg/ml dextran sulfate | 82 | 99 |
| 2 U/ml chondroitin ABC lyase | 49 | 100 |
| 4 U/ml chondroitin ABC lyase | 44 | 30 |
| 6 U/ml chondroitin ABC lyase | 43 | 0 |
| 1-6 U/ml heparitinase | 39 | 0 |
| PEG | 33 | 6 |

*0.66 mg/ml collagenase type IV was toxic to isolated myofibers

Approaches to Rescue Adult Skeletal Muscle Infectivity

Previous studies have suggested that basal lamina formation during maturation may act as a physical barrier to HSV infection, thereby preventing interaction of the virus with the receptors required for infectivity. To test this, isolated myofibers were exposed to G207 following treatment with collagenase type IV, which liberates peptides from collagen thereby degrading the basal lamina (FIG. 1). Indirect immunofluorescence of a nuclear HSV protein, ICP4, revealed that partial destruction of the basal lamina in this manner stimulated HSV infection (FIG. 1, d). The effect was concentration dependent such that an increase in collagenase type IV correlated with an increase in HSV infection. Toxicity occurred at 0.66 mg/ml as indicated by myofiber hypercontraction during the 30 minute preincubation period (Table 1). In a second approach, chondroitin ABC lyase, which degrades a broad range of chondroitin sulfate moieties, was tested for its ability to enhance susceptibility to HSV infection (FIG. 1, g and h). This treatment strongly enhanced infection, whereas treatment with heparitinase did not (Table 1). Thus, partial destruction of the basal lamina with specific enzymes allowed for the attachment and entry of HSV into the mature muscle fiber, which suggests that virus secondary receptors were present but not accessible in the context of the mature myofiber.

Analysis of Cell Surface Glycosaminoglycans

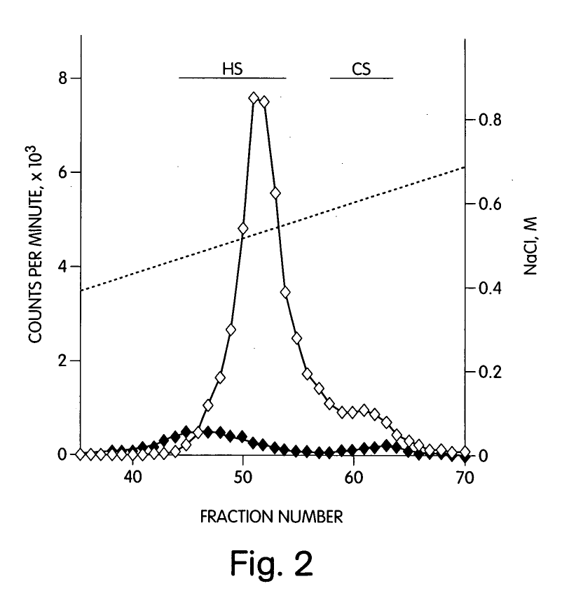

HSV infects cells by attaching to cell surface heparan sulfate-like moieties followed by interaction with secondary protein receptors (Spear et al., Adv. Exp. Med. & Biol. 313:341-353, 1992; Gruenheid et al., J. Virol. 67(1):93-100, 1993; Geraghty et al., Science 280(5369):1618-1620, 1998; Montgomery et al., Cell 87(3):427-436, 1996). Although not strictly required, cell surface heparan sulfate increases the efficiency of HSV infection by two orders of magnitude in most cells tested (Banfield et al., J. Virol. 69(9):3290-3298, 1995). To investigate whether glycosaminoglycan expression was altered in adult versus newborn muscle fibers, radiolabeled glycosaminoglycans were isolated from muscle fiber cultures and analyzed by anion-exchange HPLC (FIG. 2). Newborn muscle fibers expressed significant amounts of heparan sulfate and chondroitin sulfate glycosaminoglycans. By contrast, glycosaminoglycan synthesis was significantly reduced in adult myofibers during steady-state labeling, and the residual heparan sulfate synthesized was relatively under-sulfated compared with newborn myofibers (FIG. 2).

Dextran Sulfate Restores HSV Infection in Mature Myofibers

The data so far indicated that one or more components of the basal lamina present in mature myofibers inhibited HSV infection. Moreover, heparan sulfate biosynthesis was reduced compared with immature myofibers, which could account for all or part of the loss of susceptibility to HSV infection. It has been shown previously that cells devoid of heparan sulfate biosynthesis can be infected with HSV-1, but not HSV-2, if a low concentration of dextran sulfate is added to the cells either prior to or during infection (Dyer et al., J. Virol. 71(1):191-198, 1997). By contrast, dextran sulfate is a potent inhibitor of HSV infection if the target cells express significant amounts of heparan sulfate. When dextran sulfate was added to mature myofibers in culture, HSV-1 infection was significantly enhanced (FIG. 1, e and f, and Table 1). Moreover, this effect was specific for HSV-1, which is consistent with the hypothesis that the lack of mature myofiber infection was due, at least in part, to a lack of accessible heparan sulfate moieties on the cell surface (Table 2).

TABLE 2
Dextran sulfate stimulation of mature myofibers with
G207 (HSV-1) vs. LIBRI (HSV-2)

| | | β-galactosidase |
|---|---|---|
| Treatment | Virus | expression |
| No treatment | G207 | negative |
| | L1BR1 | negative |
| 10 μg/ml dextran sulfate | G207 | positive |
| | L1BR1 | negative |

To test whether there was an additional block in the post-attachment fusion of HSV with the plasma membrane, isolated mature myofibers were exposed to the fusogenic agent polyethylene-glycol (PEG) prior to challenge with G207. PEG-induced fusion did not alter adult myofiber infectivity, suggesting that the block to HSV infection occurred at the level of viral attachment (Table 1).

Infection of Skeletal Muscle

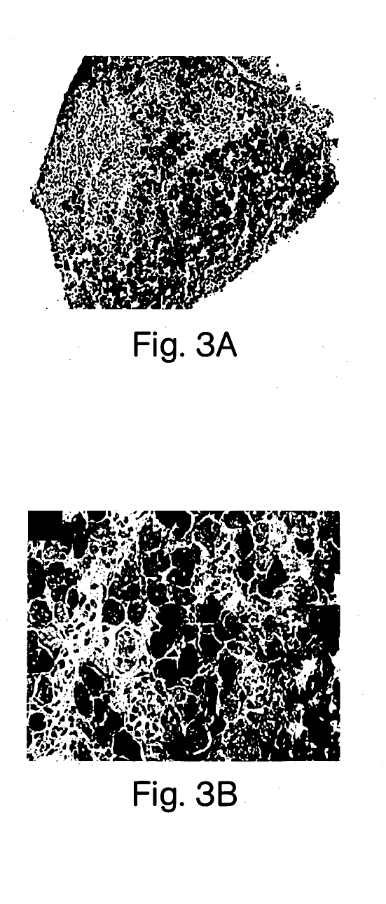

To establish that immature myofibers were susceptible to G207 infection in vivo, 10⁶ plaque forming units (pfu) were injected directly into the tibialis anterior (TA) muscle of an 8-day old balb/c mouse. Injected muscles were removed three days post-injection, sectioned, and analyzed histochemically for the expression of β-galactosidase (FIG. 3). High levels of transgene expression were detected in the injected area. HSV infected myofibers were also found away from the site of injection, which suggests that there was considerable spread of the vector.

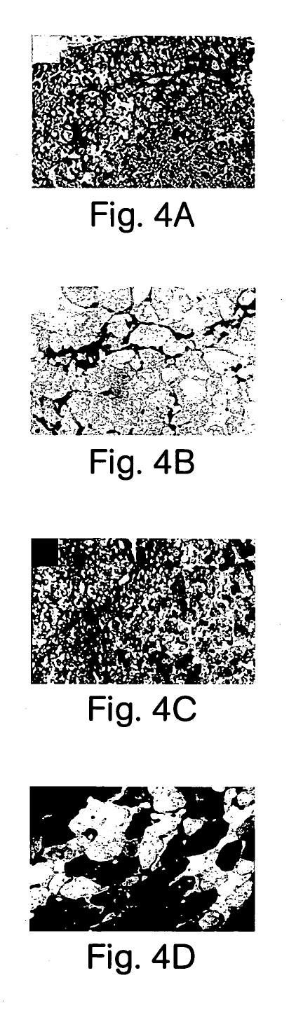

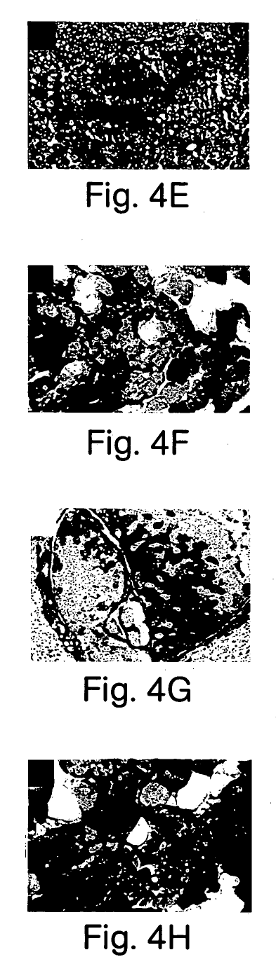

To test whether the three treatments that enhanced infection in vitro worked in the adult animal, mice were injected with 1×10⁶ pfu of G207 in the tibialis anterior (TA) muscle along with either chondroitin ABC lyase, collagenase type IV, or dextran sulfate. In all instances, the in vivo results were consistent with the observations made in vitro (FIG. 4, Table 3). Interestingly, dextran sulfate could be administered an hour prior to virus with no loss of function, an observation also made in vitro (Dyer et al., J. Virol. 71(1):191-198, 1997). In addition, infection was not limited to regenerating myofibers, which were identified by their centrally-located nuclei. Taken together, these results show that the barrier to HSV infection in adult skeletal muscle was due, at least in part, to the relative paucity of HSV receptors required for efficient infection.

TABLE 3
Number of lacZ-expressing fibers in mature mouse TA
muscle following gene transfer by intramuscular co-injection
of treatment with G207

| Treatment | # of blue fibers |
|---|---|
| G207 only | 0 |
| 3 μg/ml dextran sulfate | 76 |
| 10 μg/ml dextran sulfate | 149 |
| 0.18 mg/ml collagenase type IV | 0 |
| 0.33 mg/ml collagenase type IV | 77 |
| 2 U/ml chondroitin ABC lyase | 302 |
| 4 U/ml chondroitin ABC lyase | 226 |

HSV Toxicity After Systemic Delivery

The toxicity of HSV in mice delivered NV1020 systemically was first assessed. Mice were administered various doses of NV1020 either by an intrasplenic route, by portal vein, or by tail vein. Morbidity and mortality were assessed every day for 28 days (Table 4). These studies demonstrated that 1×10⁷ pfu can be delivered to mice through a variety of routes without any observable toxicity. This no-effect dose is equivalent to approximately 3.5×10¹⁰ pfu in humans.

TABLE 4
HSV-toxicity after vascular delivery

| Mouse | Virus | Dose (pfu) | Route | Morbidity | Mortality |
|---|---|---|---|---|---|
| C57B6 | NV1020 | 5E7 | Intrasplenic | +++ | 50% |
| Balb/C | NV1020 | 5E7 | Intrasplenic | +++ | 100% |
| Balb/C | NV1020 | 1E7 | Intrasplenic | + | 0% |
| Balb/C | NV1020 | 3E7 | Portal vein | +++ | >50%† |
| Balb/C | NV1020 | 1E8 | Tail vein | +++ | 100%* |
| Balb/C | NV1020 | 3E7 | Tail vein | − | 0% |
| Balb/C | NV1020 | 3X1E7 | Tail vein | − | 0% |

†mortality % depends upon viral purification process
*not observed if mice are > 12 weeks old

Systemic Delivery of HSV Results in Infection of Tumor Tissue

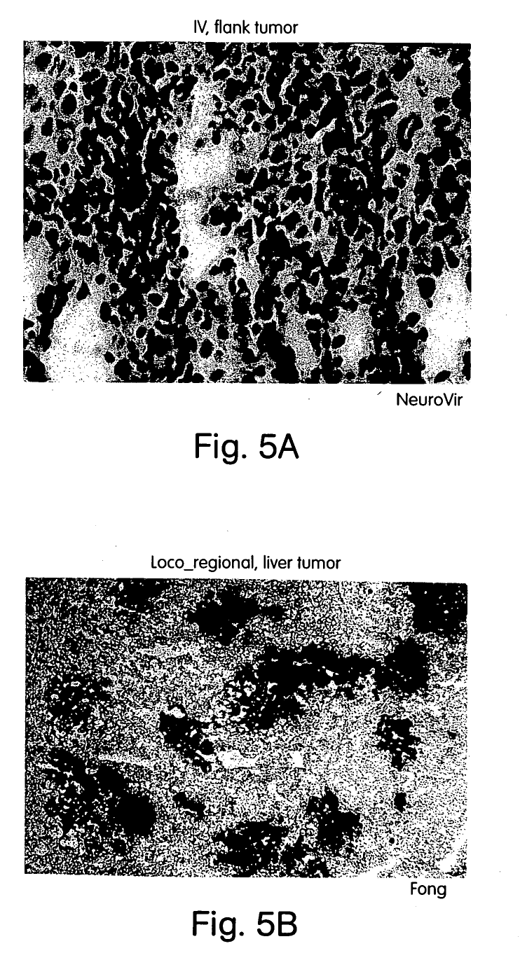

HSV can also be delivered to tumor tissue as an antitumor agent. For example, HSV was administered to mice, by tail vein (G47Δ-BAC, a derivative of G207 with ICP47 deleted and a bacterial artificial chromosome (BAC) element inserted into the thymidine kinase locus), or locoregionally (G207), by portal vein, reaching a distant flank tumor or liver tumor, respectively. The virus successfully infected the tumor cells in each of the models (FIG. 5). Viral-induced destruction of tumor cells can be followed by tumor necrosis, resulting in delayed tumor growth and regression.

Delivery of Viral Particles to Various Tissues After Systemic Administration

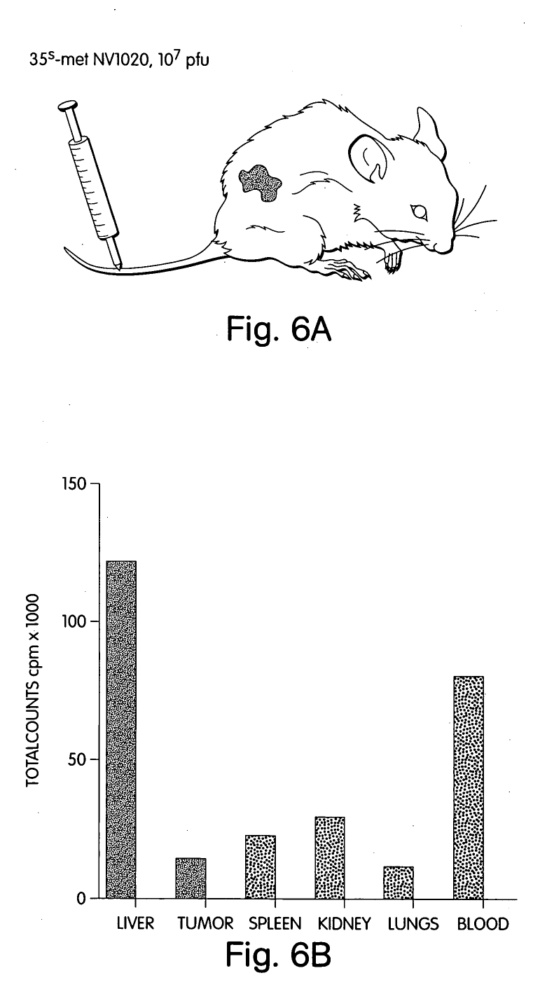

The estimated number of viral particles that reach a tumor after systemic administration was also determined. Mice with flank tumors were injected, by tail vein, with 1×10⁷ pfu of ³⁵S methionine-labeled NV1020. NV1020 is a recombinant replication competent vector containing only one copy of γ34.5 and a deletion in the terminal repeats such that rearrangement of genome segments is ablated. After 2 hours, the mice were sacrificed, and their tissues were harvested and measured for the presence of virus particles (by measuring the radioactive label). The results of these studies indicated that approximately 10³ to 10⁴ viral particles were found in the liver, and approximately 10⁴ to 10⁵ particles were located in the tumor tissue (FIG. 6).

Systemic Delivery of NV10020 Significantly Increases Survival in a CT-26 Liver Metastatic Cancer Model

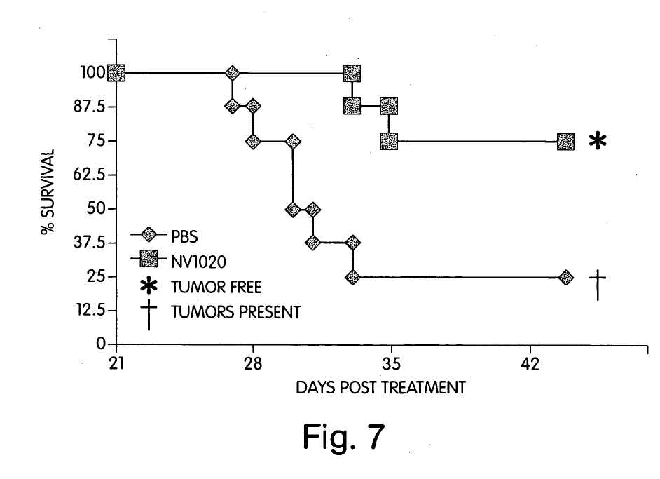

NV1020 was also used to examine the effect of administration of this viral vector on mice in a metastatic cancer model. In this model, liver tumor nodules formed in mice following an intrasplenic injection of CT-26 cancer cells. Twenty-four hours after cell injection the mice were injected, by tail vein, with 1×10⁷ pfu of NV1020. The mice were carefully monitored and assessed for survival over time (FIG. 7). Moribund animals were appropriately sacrificed. Survival was increased from 25% to 75% by the administration of NV1020, compared to control animals.

Intratumoral or Systemic Delivery of NV1020 Results in Anti-tumor Efficacy in a CT-26 Flank Tumor Model

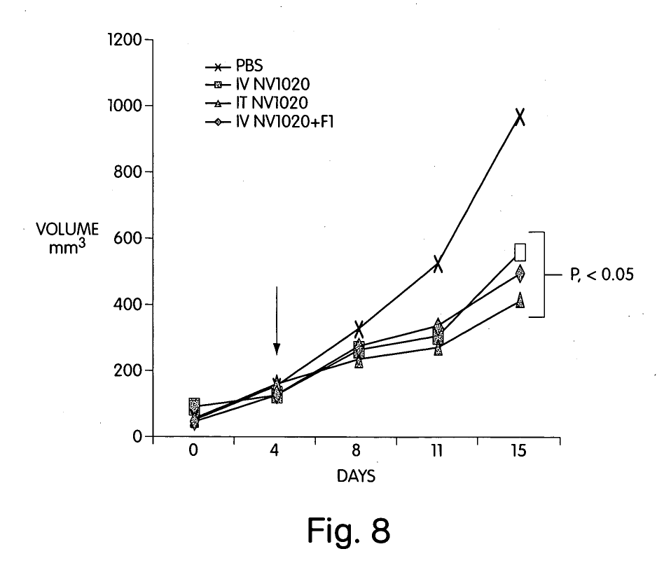

Different modes of delivery were also examined to determine the efficacy of systemic delivery of HSV on tumor growth. Flank tumors were established in mice by subcutaneous injection of CT-26 cancer cells. The mice were then administered 1×10⁷ pfu of NV1020 either intratumorally or systemically by tail vein. Tumor growth rates were then assessed by measuring the tumor volume biweekly (FIG. 8).

The results of this study indicate that intratumoral administration of NV1020 is as efficacious as systemic delivery by tail vein (no statistical difference). Both routes of administration significantly delayed the tumor growth rate, resulting in an approximately 50% reduction in tumor volume. These results demonstrate that although virus is typically administered by intratumoral injection in tumor models (because it is thought that systemic delivery of virus would not result in sufficient pfu reaching the tumor for efficacy), other routes of administration are also effective.

The addition of a glycosaminoglycan analog to the viral vector therapy for tumor treatment was also investigated. Dextran sulfate is a glycosaminoglycan analog that is used in a research setting to block viral infection of target cells by interfering with viral attachment to cells. In a clinical setting, it is used for local perfusion of therapeutics following surgery. Dextran sulfate is also reported to be a volume expander. Most recently, dextran sulfate has been tested as an antiviral agent for HIV.

The CT-26 flank tumor model was again used to determine the effect of a combination of HSV and dextran sulfate analog on tumor growth. A combination of 1×10⁷ pfu of NV1020 and dextran sulfate (100 μg/ml) was administered, by tail vein, to mice with flank tumors, and the tumor volume was assessed over time. NV1020 plus dextran decreased the tumor volume as well as NV1020 alone (FIG. 8).

Anti-tumor Efficacy Increases with Dose of Dextran Sulfate Administered

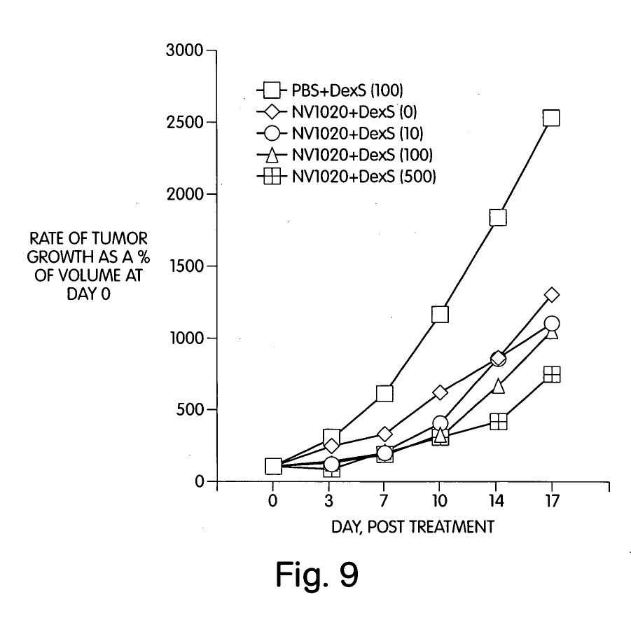

The effect of the concentration of charged molecule administered with HSV on tumor growth was also evaluated. Mice with flank tumors were administered one dose of 1×10⁷ pfu of NV1020, 1×10⁷ pfu of NV1020 plus 10 μg/ml of dextran sulfate, 1×10 pfu of NV1020 plus 100 μg/ml of dextran sulfate (100 μg/ml), 1×10⁷ pfu of NV1020 plus 500 μg/ml of dextran sulfate, or PBS plus 100 μg/ml of dextran sulfate (control) at day 0, day 2, and day 4. Tumor growth rates were then assessed (FIG. 9). These studies revealed that dextran sulfate enhanced viral therapy in a dose-dependent manner, with higher concentrations of dextran sulfate co-administered with NV1020 being more efficacious than lower concentrations.

Multiple Dosing Increases Anti-tumor Efficacy in a CT-26 Flank Tumor Model

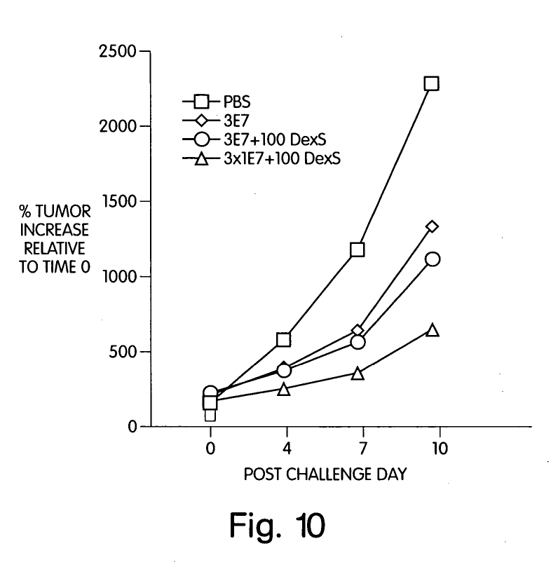

The effect of multiple doses of HSV plus dextran sulfate on tumor growth was also evaluated. Mice with flank tumors were administered three doses of 1×10⁷ pfu of NV1020, one dose of 3×10⁷ pfu of NV1020, three doses of 1×10⁷ pfu of NV1020 plus 100 μg/ml of dextran sulfate (F1), or PBS (control). Tumor growth rates were then assessed (FIG. 10).

The results of this study showed that tumor growth was lower when 100 μg/ml of dextran sulfate was co-injected with NV1020. In addition, anti-tumor efficacy is increased when three doses of 1×10⁷ pfu of NV1020 were administered compared to when one dose of 3×10⁷ pfu of NV1020 was administered.

Multiple Dosing of NV1020 with Dextran Sulfate Increases Survival in a CT-26 Flank Tumor Model

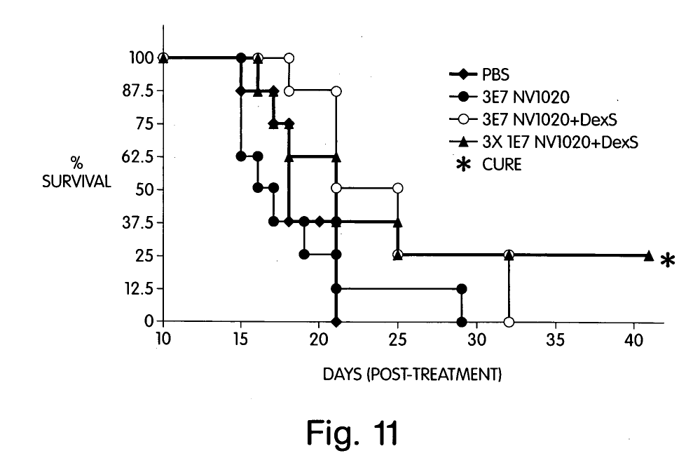

Many reports of anti-tumor efficacy do not result in a corresponding increase in subject survival. To determine if anti-tumor efficacy corresponds to an increase in survival in animals treated with HSV or HSV plus dextran sulfate, mice with flank tumors were administered one dose of 3×10⁷ pfu of NV1020, one dose of 3×10⁷ pfu of NV1020 plus 100 μg/ml of dextran sulfate, three doses of 1×10⁷ pfu of NV1020 plus 100 μg/ml of dextran sulfate, or PBS (control). Mouse survival was then measured over time (FIG. 11). These studies showed that NV1020, delivered in multiple doses along with dextran sulfate, increased the survival of mice. These results correspond to the anti-tumor efficacy of NV1020 plus dextran sulfate described above. With such a treatment, cures can result.

Dextran Sulfate Increases Efficacy in a CT-26 Liver Metastatic Model

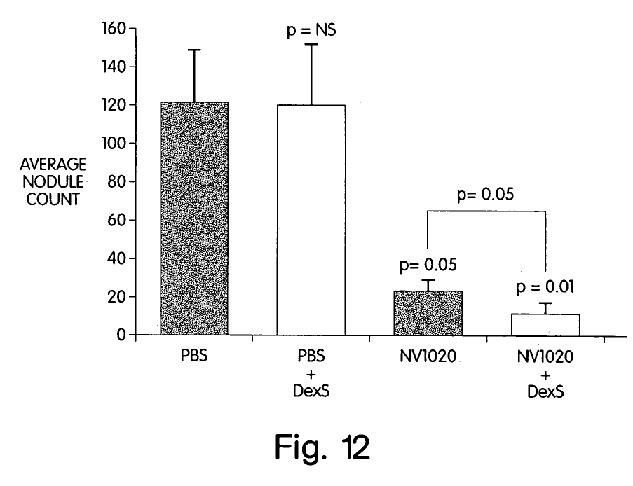

The efficacy of formulation changes was also evaluated in a mouse CT-26 liver metastatic model. Mice with metastasized tumors were administered 1×10⁷ pfu of NV1020, 1×10⁷ pfu of NV1020 plus dextran sulfate (100 μg/ml), PBS only (control), or PBS plus dextran sulfate (100 μg/ml)(control). The number of tumor nodules was then assessed 13 days after treatment (FIG. 12). Treatment with each of the three formulations resulted in decreased nodule counts compared to controls. These results establish that this treatment protocol is not only effective in the instance of single, large, established flank tumors, but also in the instance of microscopic disease.

Dextran and Acyclovir also Increase Anti-tumor Efficacy in a CT-26 Flank Tumor Model

To examine the roles of sulfation of dextran sulfate and the replication of HSV in the anti-tumor efficacy of HSV plus dextran sulfate formulations, dextran or acyclovir were added to the formulations. The molecule dextran has been used in the clinic as a volume expander and for local perfusion of tissue following surgery. There are also reports of using dextran for routine cardiovascular therapy in Japan. Acyclovir is a clinically approved drug used to prevent replication and hence inhibit the spread of HSV. It is an obligate chain terminator activated by viral thymidine kinase.

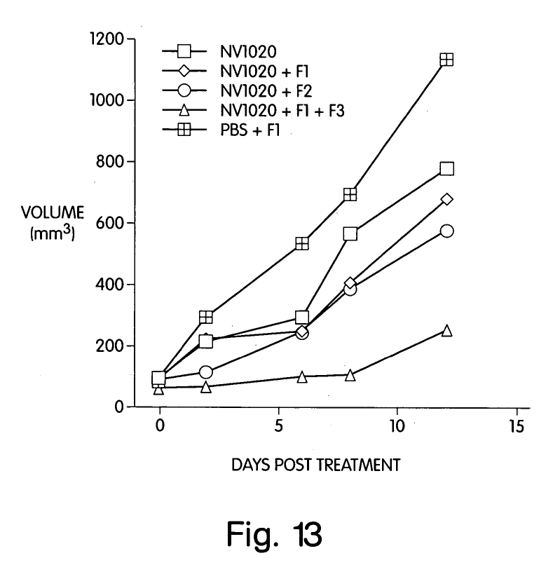

Mice with flank tumors were administered 1×10⁷ pfu of NV1020, 1×10⁷ of pfu NV1020 plus dextran sulfate (100 μg/ml), 1×10⁷ pfu of NV1020 plus dextran only, 1×10⁷ pfu of NV1020 plus dextran sulfate (100 μg/ml) and acyclovir (2 mg/ml), or PBS plus dextran sulfate (100 μg/ml). Tumor growth rates were then assessed biweekly (FIG. 13). The sulfate component of dextran sulfate did not appear to affect its mode of increasing anti-tumor efficacy, as co-injection of HSV with dextran alone gave a comparable anti-tumor efficacy. As well, viral replication does not appear to be necessary for anti-tumor efficacy or mouse survival, as the combination of HSV, dextran sulfate, and acyclovir reduced tumor growth.

G207 Anti-tumor Efficacy is Enhanced by Dextran Sulfate

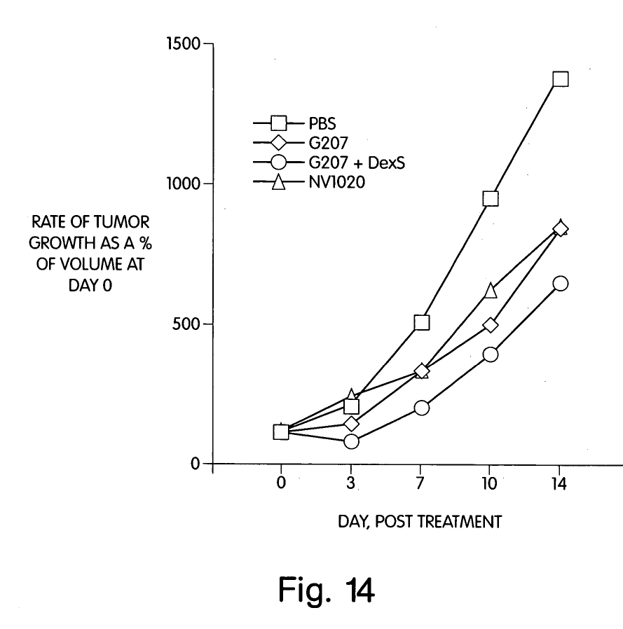

Another recombinant HSV vector, G207, a different strain than NV1020 with both copies of γ34.5 deleted and inactivation of ribonucleotide reductase by insertion of the β-galactosidase gene was also tested for its anti-tumor efficacy. Mice with flank tumors were administered 1×10⁷ pfu of NV1020, 1×10⁷ pfu of G207, 1×10⁷ pfu of G207 plus dextran sulfate (100 μg/ml), or PBS plus dextran sulfate (100 μg/ml)(control) at day 0, day 2, and day 4. Tumor growth rates were then measured (FIG. 14). Dextran sulfate increased anti-tumor efficacy of G207, indicating that other strains of HSV vectors are of therapeutic value, and that HSV-1 anti-tumor efficacy is not dependent on γ34.5. These findings also suggest that other current generation vectors may also be used therapeutically.

Dextran Sulfate Alters CT-26 Morphology in vitro, but Not Cell Growth

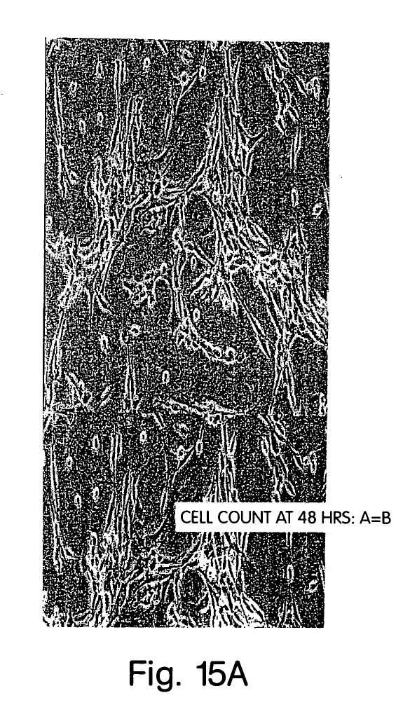

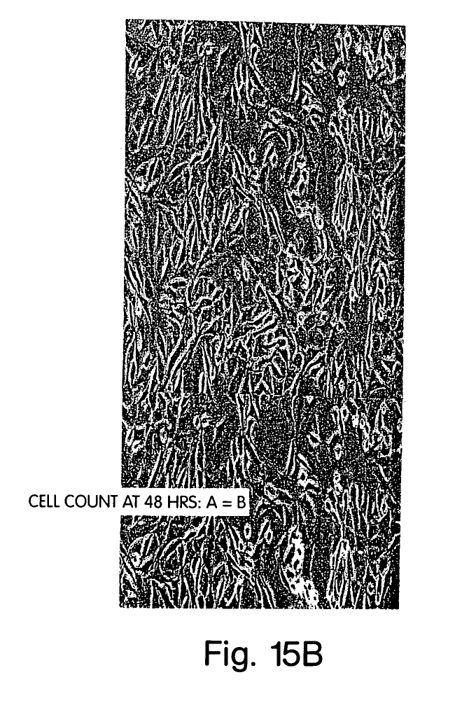

To better understand the in vivo effects of dextran sulfate or dextran in combination with HSV, the effects of dextran sulfate on cell morphology and cell proliferation were evaluated in a cell culture model. CT-26 cells were treated with 100 μg/ml of dextran sulfate or were left untreated. After 48 hours, cell numbers and morphology were examined (FIG. 15). In vitro, dextran sulfate added to cell culture media did not slow the growth of CT-26 cells. Treatment of the cells with dextran sulfate did, however, change the morphology of the cells. In the presence of dextran sulfate, CT-26 cells appeared more evenly spread across tissue culture dishes (panel B) as compared to the “clumped” appearance of CT-26 cells in the absence of dextran sulfate (panel A). These results suggest that a change in gene expression may account for the profound anti-tumor efficacy seen in vivo.

Dextran Sulfate and Acyclovir do Not Affect CT-26 Growth in Culture

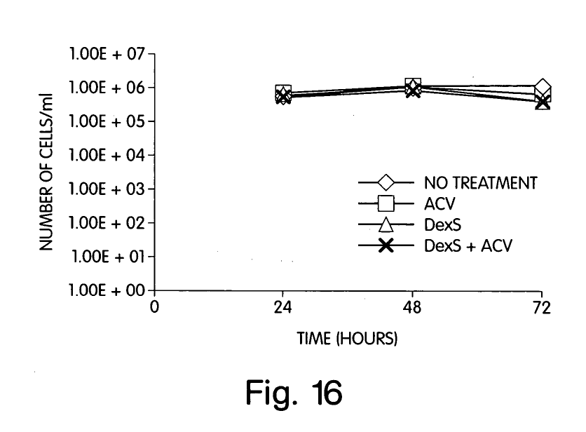

The effect of dextran sulfate, acyclovir, or a combination of both dextran and acyclovir were also examined for their effects on cell growth in vitro. Cultured CT-26 cells were administered dextran sulfate (100 μg/ml), acyclovir (2 mg/ml), both dextran sulfate and acyclovir, or were left untreated (control). CT-26 cells were counted at various time points following addition each formulation. After 72 hours of exposure to the formulations, the growth of the cells (number of cells/ml) did not vary significantly upon exposure to dextran sulfate, acyclovir, or a combination of both formulations, compared to untreated cells (FIG. 16). Such results indicate that these formulations do not alter cell growth in vitro.

Dextran Sulfate Increases Peripheral Degeneration of Tumors

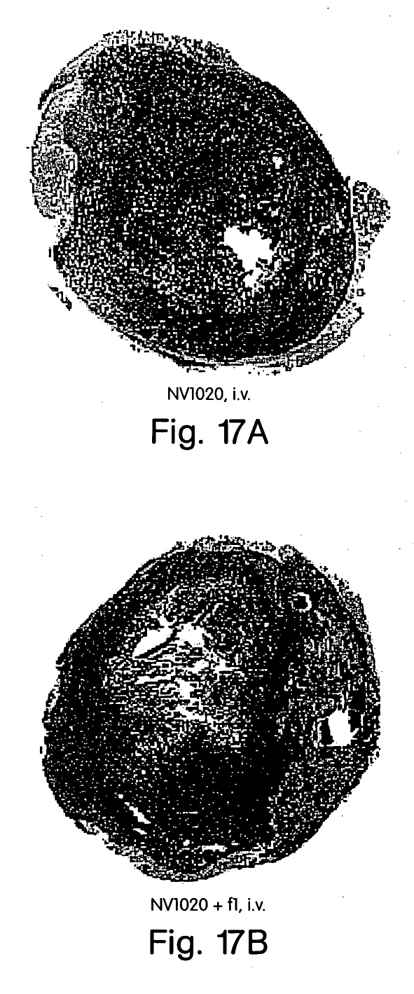

The result of HSV co-injected with dextran sulfate on tumor degeneration was examined next. Mice with flank tumors were administered NV1020 or NV1020 plus dextran sulfate (F1) by tail vein. The tumor was then removed, frozen, and sectioned for histochemical analysis (FIG. 17). Peripheral tumor degeneration was much greater when dextran sulfate was administered with NV1020 (panel B; area shown in light purple), than when NV1020 was administered alone (panel A). This indicates that tumor necrosis is not limited to anoxic cells at the center of the tumor, but also to growing and dividing tumor cells located that the margin where blood vessels feeding the tumor are located.

Dextran Sulfate Increases the Bioavailability of Virus in vivo

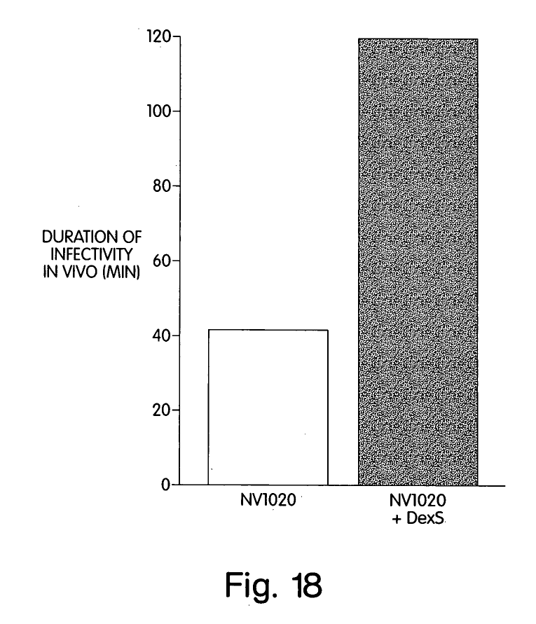

As it is known that virus is quickly inactivated by components present in blood, including those of the immune system (complement factors, antibodies), the effect of dextran sulfate on the bioavailability of virus in vivo was examined. One×10⁷ pfu of NV1020 or 1×10⁷ pfu of NV1020 plus dextran sulfate (100 μg/ml) was administered by tail vein to mice. Groups of mice were then sacrificed at varying time points. Blood samples were removed by heart puncture, and serum was analyzed to determine the length of time in which the virus was infective in vivo (FIG. 18). When dextran sulfate was administered with NV1020, the circulation time of infectious virus was increased by 3-fold. Such a time is sufficient to enable the active virus to reach a tumor in order to mediate anti-tumor efficacy. Accordingly, a longer circulation time of infectious virus results in a greater net therapeutic vector delivered.

Dextran Sulfate Alters the in vivo Distribution of NV1020 Such that More Virus Reaches Tumor Tissue

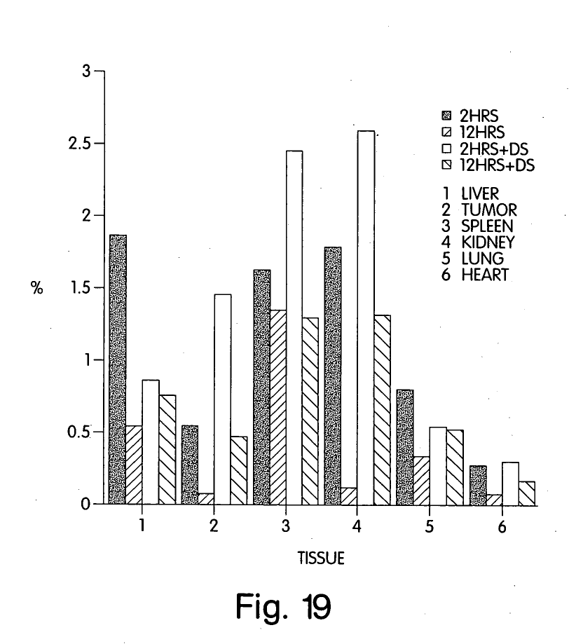

In addition, the effect of dextran sulfate on the in vivo tissue distribution of NV1020 was determined. One×10⁷ pfu of radioactive-labeled (³⁵S) NV1020 or 1×10⁷ pfu of radioactive-labeled (³⁵S) NV1020 with dextran sulfate (100 μg/ml) was administered to mice with flank tumors by tail vein. At 2 hours or 12 hours, various organs, including liver, spleen, kidney, lung, kidney, lung, and heart, as well as the tumor were harvested, homogenized, and assessed for viral load by scintillation counting (FIG. 19). Over time, dextran sulfate increased the amount of virus found in the tumor and decreased the amount of virus found in the liver.

Dextran Sulfate Decreases Angiopoiesis Factor Gene Expression

Gene expression studies in CT 26 cells treated with dextran sulfate were also completed. Cultured CT-26 cells were treated for 1 hour with dextran sulfate, or were left untreated. RNA was then extracted from the cells. Expression of a number of different genes was then measured, and the expression levels between the cells treated with dextran sulfate and untreated cells were compared. The results of these studies are summarized in Table 5. Notably, expression of a number of genes encoding proteins involved in angiopoiesis was decreased.

TABLE 5
Gene array analysis

| Gene | Function | Change |
|---|---|---|
| Flk-1 | Angiogenesis | ↓ |
| VEGF-β | Angiogenesis | ↓ |
| Endothelin R type | Angiogenesis | ↓ |
| MMP-8 | Protease | ↓↓↓ |
| TNF-α | TNF super family | ↓ |
| TRAIL-R1 | TNF super family | ↓↓↓ |
| OX-40L | TNF super family | ↑ |
| IL-18 | Cytokine | ↓↓↓ |
| Ephrin β4 | Ephrin receptor | ↓↓↓ |
| CD-6 | Cell surface | ↑ |
| α-tubulin | Housekeeping | ⇄ |

Materials and Methods

Materials

Dextran sulfate with a molecular weight of 500,000 was purchased from Pharmacia (catalog no. 17-0340-01). Chondroitin ABC lyase was purchased from Seikagaku Corporation (catalog no. 100330). Heparitinase was purchased from Seikagaku Corporation (catalog no. 100703). Collagenase type IV was purchased from Sigma (catalog no. C 1889). All tissue culture reagents (Gibco) and dishes (Nunc) were obtained from Canadian Life Technologies (Burlington, Ontario, Canada).

Viral Stocks

Recombinant NV1020, HSV-1, G207 (NeuroVir Inc.) and HSV-2, L1BR1 (Asano et al., J. Gen. Virol. 80(1):51-56, 1999; Nishiyama et al., Virology 190(1):256-268, 1992) were prepared on Vero cells. The recombinant HSV vector, NV1020, a replication competent vector contains only one copy of γ34.5 and has a deletion in the terminal repeats such that rearrangement of genome segments is ablated. G207 contains the β-galactosidase gene inserted in-frame in the ribonucleotide reductase gene. As such, this recombinant virus is unable to replicate in non-dividing cells (e.g., muscle cells). L1BR1 contains the β-galactosidase gene inserted into the US3 protein kinase gene. Virus was concentrated by centrifugation through a 30% sucrose pad, suspended in phosphate buffered saline (PBS), and filtered through a 0.45 μm filter (Sartorius), using standard methods. The final titer of infectious virus used for all experiments was 1×10⁸ pfu/ml.

Primary Muscle Fiber Cultures

Balb/c mice were bred in institutional animal care facilities at the University of British Columbia. Two different age groups were designated to be “newborn” and “adult.” The “newborn” mice were 7 to 10 days old. The “adult” mice were 6 to 12 weeks old.

Single isolated myofibers were prepared from dissected extensor digitorum longus (EDL) muscle. The myofibers were dissociated by enzymatic disaggregation in 0.2% type 1 collagenase (Sigma), followed by mild trituration. Isolated myofibers were then plated into several 24 well dishes coated with 1 mg/ml of Matrigel (Collaborative Biomedical Products). Culture medium consisting of 10% horse serum and 10% FBS in DMEM was added to the wells. These plates were then incubated for 18 hours at 37° C., at which point viable myofibers were infected with G207.

Infection of Myofibers

Myofibers were infected by adding G207 (10⁶ pfu) in culture medium (10% FBS in DMEM) directly to the wells. Incubation length was overnight (approximately 18 hours), although a one hour infection in DMEM only was sufficient to give reproducible infection. Following incubation, myofibers were fixed for 15 minutes in 1.25% glutaraldehyde and stained with 2% X-gal substrate (1 mM MgCl₂, 5 mM K₄Fe(CN)₆/K₃Fe(CN)₆ in PBS) (Canadian Life Technologies) for 4 hours at 37° C.

Indirect Immunofluorescence

Isolated myofibers were plated onto glass coverslips and infected with G207 as described above. They were then fixed in 1.25% glutaraldehyde in PBS for 15 minutes, rinsed twice with PBS, followed by 15 minutes incubation in the blocking solution (PBS with 1% bovine serum albumin (Boehringer Mannheim)). After blocking, myofibers were permeabilized with 0.1% Triton-X100/PBS for 5 minutes and incubated with a mouse anti-ICP4 antibody at 1:2000 for 1 hour. Myofibers were washed with three changes of PBS, then incubated with goat anti-mouse IgG conjugated to Texas-Red (Jackson Immunochemicals) diluted 1:200 in PBS-1% BSA for 30 minutes. The myofibers were then rinsed with PBS and mounted on glass slides. Immunofluorescence staining was observed using a BioRad MRC 600 confocal epifluorescence microscope. Confocal images were rendered using NIH Image Version 1.60 and colorized with Adobe Photoshop Version 4.0 (Adobe Systems Inc.). Standard control experiments were performed, including incubation with the secondary antibody only and with mock infected cells. All fixation and antibody incubations were performed at RT.

In vitro Treatment Assays

Assays for dextran sulfate stimulation, collagenase type IV, chondroitin ABC lyase, and heparitinase were performed on adult myofibers plated in 24 well dishes. The myofibers were pretreated with varying concentrations of dextran sulfate, collagenase type IV, chondroitin ABC lyase, or heparitinase in DMEM for 30 minutes prior to infection. After an overnight adsorption period (approximately 18 hours) at 37° C., the inoculum was removed. The myofibers were then fixed for 15 minutes in 1.25% glutaraldehyde and stained with 2% X-gal substrate for 4 hours at 37° C. For all in vitro studies, a minimum of 40 myofibers was tested per treatment group unless otherwise specified. PEG-induced fusion was performed according to methods described previously (Meyer et al., J. Gen. Virol. 79(8):1983-1987, 1998).

Analysis of Glycosaminoglycans

Biochemical labeling of glycosaminoglycans was performed by a modification of procedures described previously (Bame et al., J. Biol. Chem. 264:8059-8065, 1989). Briefly, glycosaminoglycans were radiolabeled by incubating cells for 24 hours with [³⁵S] sulfate (carrier free, approximately 43 Ci/mg, ICN) per ml in DMEM/10% FBS/10% horse serum modified to contain 10 μM sulfate. The cells were washed three times with cold PBS and solubilized with 1 ml of 0.1 N NaOH at RT for 15 minutes. Samples were removed for protein determination. Extracts were adjusted to pH 5.5 by the addition of concentrated acetic acid and treated with protease (Sigma; 2 mg/ml) in 0.32 M NaCl 40 mM sodium acetate, pH 5.5, containing shark cartilage chondroitin sulfate (2 mg/ml) as carrier, at 40° C. for 12 hours. For some experiments, portions of the radioactive material were treated for 12 hours at 40° C. with 10 mU of chondroitin ABC lyase (Sigma) or 0.5 U of heparitinase (Sigma). The radioactive products were quantified by chromatography on DEAE-Sephacel (Pharmacia) by binding in 50 mM NaCl followed by elution with 1 M NaCl. For high pressure liquid chromatography (HPLC) analysis, the glycosaminoglycan samples were desalted by precipitation with ethanol. Following centrifugation, the ethanol precipitates were suspended in 20 mM Tris (pH 7.4) and resolved by anion-exchange HPLC, using TSK DEAE-35W column (15 by 75 mm; Beckman instruments). Proteoglycans were eluted from the column by using a linear 50 to 700 mM NaCl gradient formed in 10 mM KH₂PO₄ (pH 6.0). All buffers contained 0.2% Zwittergent 3-12 (Calbiochem). The glycosaminoglycans in the peaks were identified by digestion of the sample with the relevant enzymes prior to chromatography.

Flank Tumor Model

Mice were anesthetized using ketamine (70 mg/kg) and xylazine (10 mg/kg). CT-26 cells (5×10⁴ cells resuspended in 100 μl of PBS) were injected subcutaneously into the right flank of each mouse, using a 26-gauge needle. The cells formed a tumor, which was allowed to grow to a size of approximately 100 to 150 mm³. Injections of the desired therapy were then initiated. Tumor volumes were generally measured biweekly. The animals were sacrificed once the tumor volume reached 1500 mm³.

Metastatic Cancer Model

The metastatic cancer mode has been described by Lafreniere and Rosenberg (J. Natl. Cancer Inst. 76(2):309-322, 1986).

Intramuscular Administration of the Recombinant HSV Vector

Adult and newborn mice under anesthesia (Ketamine/Rhompun intraperitoneally) were injected percutaneously into the tibialis anterior muscle (TA) to an approximate depth of 2.0 mm using a Hamilton syringe. For in vivo assays involving co-injection of treatment solutions along with the viral inoculum, dextran sulfate, collagenase type IV, or chondroitin ABC lyase was diluted to the appropriate concentration (as identified by in vitro studies) with G207 in an injection volume of 50 μl (for adult mice) or 25 μl (for newborn mice). Control muscles were injected with G207 only. To evaluate myofiber infection, muscles were removed 3 days post-injection for sectioning and histological analysis. For any of the procedures, a minimum of 4 animals received identical treatment and comprised an experimental group.

Tail Vein Administration of the Recombinant HSV Vector

Tail vein administration of the desired therapy was carried out using techniques commonly known in the art.

Tissue Sectioning

The injected and control muscle or tumor tissue were rapidly frozen. The muscles or tumor tissue were sectioned, yielding serial cross-sections throughout the tissue. Cross-sections (10 μm) were cut on a cryostat and stained with X-gal and/or hematoxylin and eosin. The sections were retained at regular intervals (approximately every 120 μm). For histology, the cryosections were collected onto gelatin-coated glass slides.

Histological Analysis

The histological detection of β-galactosidase-expressing cells in cryosections was done using X-gal. This compound yields a blue reaction product in cells expressing high levels of β-galactosidase. The sections were first fixed by dipping the slides in 4% paraformaldehyde in 100 mM NaP, pH 7.2, for 5 minutes. The slides were rinsed three times for 5 minutes in PBS. The sections were then stained with X-gal (Sigma) at a concentration of 1 mg/ml in 5 mM K₃Fe(CN)₆, 5 mM K₄Fe(CN)₆, 2 mM MgCl₂ in PBS for 12 hours. The slides were mounted using an aqueous mounting medium (Promount) and examined microscopically for the presence of β-galactosidase-labeled (“blue”) myofibers. The total number of lacZ-expressing fibers in a muscle was determined from the section with the maximal number of blue fibers, and that was invariably at the site of implantation.

Alternatively, cells infected with HSV were detected using standard immunohistochemical procedure and an antibody that recognizes HSV antigen.

Determining the Bioavailablity of Virus in vivo

The serum from blood obtained from mice infected with NV1020 was applied to cultured cells. The duration of time for which the virus was able to infect the cells was determined as described above.

All publications mentioned herein are hereby incorporated by reference in their entirety.

What is claimed is:

1. A method for introducing a nucleic acid vector into a living cell, said method comprising contacting said cell with said vector and, either before, during, or after contacting said cell with said vector, contacting said cell with a liquid medium comprising a compound that, in said medium, is charged, non-cytotoxic, and capable of facilitating the uptake of the vector by the cell.

2. The method of claim 1, wherein said cell is in a mammal.

3. The method of claim 3, wherein said mammal is a human patient.

4. The method of claim 1, wherein said vector comprises a gene encoding a polypeptide, a hormone, a vaccine antigen, an antisense molecule, or a ribozyme.

5. The method of claim 4, wherein said polypeptide is selected from the group consisting of growth factors, enzymes, anti-angiogenic polypeptides, and polypeptides that promote cell death.

6. The method of claim 1, wherein said vector is a viral-based vector.

7. The method of claim 6, wherein said vector is selected from the group consisting of a Herpesviridae, Dengue, Adeno-associated virus, Adenovirus, papillomavirus, and retrovirus based vectors.

8. The method of claim 7, wherein said vector is selected from the group consisting of HSV-1, HSV-2, VZV, CMV, EBV, HHV-6, HHV-7, and HHV-8.

9. The method of claim 7, wherein said vector is a lentivirus-based vector.

10. The method of claim 9, wherein said vector is an HIV-based vector.

11. The method of claim 1, wherein said vector is a bacterial vector.

12. The method of claim 11, wherein said vector is a Listeria monocytogenes-based vector.

13. The method of claim 1, wherein said vector is attenuated.

14. The method of claim 1, wherein said charged molecule is selected from the group consisting of charged polysaccharides, polylysine, acyclodextrin, diethylaminoethane, and polyethylene glycol.

15. The method of claim 14, wherein said charged polysaccharide is a glycosaminoglycan.

16. The method of claim 14, wherein said charged polysaccharide is a glycosaminoglycan analog.

17. The method of claim 15, wherein said glycosaminoglycan is selected from the group consisting of dermatan sulfate, heparan sulfate, chondroitin sulfate, and keratin sulfate.

18. The method of claim 16, wherein said glycosaminoglycan analog is dextran sulfate.

19. The method of claim 1, wherein said charged molecule is administered to said cell prior to the administration of said vector to said cell.

20. The method of claim 1, wherein said charged molecule is administered to said cell concurrent with the administration of said vector to said cell.

21. The method of claim 1, wherein said cell is a mature muscle cell.

22. The method of claim 3, wherein said cell is a cancer cell.

23. The method of claim 22, wherein said patient has cancer.

24. The method of claim 21, wherein said muscle cell is in a patient with a primary myopathy.

25. The method of claim 3, wherein said patient has a condition that can be treated by production of a therapeutic product for secretion into said subject's circulation.

26. The method of claim 3, wherein said vector and charged molecule are delivered locally.

27. The method of claim 3, wherein said vector and charged molecule are delivery systemically.
